(19) United States
(12) Patent Application Publication
(10) Pub. No.: US 2021/0106702 A1
(43) Pub. Date: Apr. 15, 2021

(54) NEUTRON BEAM DYNAMIC THERAPY

(71) Applicant: DYNAMIC SCIENCE CORPORATION, Chuo-ku (JP)

(72) Inventors: Hitoshi HORI, Chuo-ku (JP); Ken TOKUNAGA, Chuo-ku (JP); Masao TAKAHASHI, Chuo-ku (JP)

(73) Assignee: DYNAMIC SCIENCE CORPORATION, Chuo-ku (JP)

(21) Appl. No.: 16/982,155

(22) PCT Filed: Sep. 19, 2018
(86) PCT No.: PCT/JP2018/035822

§ 371 Date: Dec. 21, 2020

(30) Foreign Application Priority Data
Mar. 19, 2018 (JP) 2018-072735

(51) Int. Cl. A61K 51/04; A61K 41/00; C07B 59/00

(52) U.S. Cl. CPC A61K 51/0491; C07B 2200/05; C07B 59/005; A61K 41/0095

(57) ABSTRACT

Provided is a new boron isotope (¹⁰B) tracing drug which can exhibit wide-area molecular-targeting function and stereotactic destructive power. The invented boron isotope (¹⁰B) tracing drug has higher selective tumor-targeting and tumor-accumulating pharmacokinetic functions appropriate for neutron beam dynamic therapy (NBDT) and boron neutron capture therapy (BNCT).

### FIELD OF THE INVENTION

This invention relates to a creation of a boron isotope (¹⁰B) tracing drug having molecular-targeting function for neutron beam dynamic therapy (NBDT), named NBDT drug. The invented boron isotope (¹⁰B) tracing drug with its molecular-targeting function and stereotactic destructive power can be used also for boron neutron capture therapy (BNCT).

### BACKGROUND OF THE INVENTION

The invented neutron beam dynamic therapy (NBDT) is characterized by a boron isotope (¹⁰B) tracing drug which exhibits a wide-area molecular-targeting function and stereotactic destructive power by neutron irradiation. The NBDT drug is completely unique among compounds conventionally developed as boron carriers for boron neutron capture therapy (BNCT) since the NBDT drug itself exhibits pharmacological activities in advanced radiation oncology.

The NBDT according to the present invention is a boron isotope compound which can exhibit molecular-targeting function and stereotactic destructive power under neutron irradiation and can be applied also to the molecular-design for hypoxic cell radiosensitizers, hypoxia-targeting drugs, and hypoxia imaging drugs.

The NBDT according to the present invention can apply all drugs containing a boron isotope (¹⁰B) to become pharmacokinetic tracers. Thus new and previous clinical drugs can be changed as tracing agents themselves without their further syntheses of their structures incorporated stable-isotopes and radioisotopes using chemical doping techniques incorporated with boron isotope (¹⁰B), as an application of NBDT.

The present invention can be applied for the drug delivery system (DDS) of cancer treatment, in which the boron isotope (¹⁰B) tracing drug used for NBDT is made to reach the tumor area and shows its molecular-targeting function and stereotactic destructive power in the tumor area.

### PRIOR ART LITERATURES

### Non-patent documents

Non-patent document 1: Hari H, Uto Y, Nakata E. Medicinal elecronomics bricolage design of Hypoxia-targeting antineoplastic drugs and invention of boron trace drugs as innovative future-architectural drugs Anticancer Res., 30, 3233-3242 (2010).
Non-patent document 2: Masunaga S, Nagasawa H, Sakurai Y, Uto Y, Hori H, Nagata K, Suzuki M, Maruhashi A, Kinashi Y, Ono K. Int. J. Hyperthermia, 22, 287-299(2006).
Non-patent document 3: Nakata E Nagasawa H, Gotoh K, Sakurai Y, Uto Y, Hori H, Nagata K, Suzuki M, Maruhashi A, Kinashi Y, Ono K. Radiation Medicine, 24, 98-107 (2006).
Non-patent document 4: Masunaga S, Nagasawa H, Hiraoka M, Sakurai Y, Uto Y, Hori H, Nagata K, Suzuki M, Maruhashi A, Kinashi Y, AnticancerRes., 24, 2975-2984 (2004).
Non-patent document 5: Masunaga S, Nagasawa H, Hiraoka M, Sakurai Y, Hori H, Nagata K, Suzuki M, Maruhashi A, Kinashi Y, Ono K. Appl. Radiat. Isot., 61, 953-958 (2004)
Non-patent document 6: Nakata E, Yukimachi Y, Nazumi Y, Uto Y, Maezawa H, Hashimoto T, Okamoto Y, Hori H. Chem. Commun., 46, 3526-3528 (2010)
Non-patent document 7: Nakashima H, Ikkyu K, Nakashima K, Sano K, Nakata E, Nagasawa H, Sugimoto H, Shiro Y, Nakagawa Y, Hori H. Adv. Exp. Med. Biol., 662, 315-321(2010)
Non-patent document 8: Nakata E, Yakimachi Y, Kariyazono H, Im S, Abe C, Uto Y, Maezawa H, Hashimoto T, Okamoto Y, Hori H. Bioorg. Med. Chem, 17, 6952-6958 (2009)
Non-patent document 9: Nakashima H, Uto Y, Nakata E, Nagasawa H, Ikkyu K, Hiraoka N, Nakashima K, Sasaki Y, Sugimoto H, Shiro Y, Hashimoto Okamoto Y, Asakawa Y, Hori H. Bioorg. Med. Chem. 16, 8661-8669(2008)
Non-patent document 10: Nakayama S, Uto Y, Tanimoto K, Okuno Y, Sasaki Y, Nagasawa H, Nakata E, Araki K, Momose K, Fujita T, Hashimoto T, Okamoto Y, Asakawa Y, Goto S, Hori H. Bioorg. Med. Chem. 16, 7705-7714 (2008)
Non-patent document 11: Uto Y, Nagasawa H, Jin C.-Z., Nakayama S, Tanaka A, Kiyoi S, Nakashima H, Shimamura M, Inayama S, Fujiwara T, Takeuchi Y, Uehara Y, Kirk K. L., Nakata E, Hori H. Bioorg. Med. Chem. 16, 6042-6053 (2008)
Non-patent document 12: Vijay Kumar G. R., Manohar B., Divakar S. Eur. Food Res. Technol. 223,725-730 (2006).

### SUMMARY OF THE INVENTION

### Problems to be Solved by the Invention

The invented boron tracing drug can be synthesized from the known drug's chemical structure embedded boron isotope (¹⁰B) to its scaffold structure, the candidate of which involves mainly benzene isosteres, such as azaborines and heterocycles, to make higher selective tumor-targeting and tumor-accumulating and higher potential DDS drugs.

### Means to Solve Problems

The present invention, NBDT, offers the boron isotope (¹⁰B) tracing drug or compound which can exhibit wide-area molecular-targeting function and stereotactic destructive power under neutron irradiation. The diligent study in solving the problem resulted in the starting material: (1E,6E)-1,7-bis(4-hydroxy-3-methoxyphenyl)-1,6-heptadiene-3,5-dione (curcumin) as shown in the formula (1) as the starting material which is the lead compounds constituting the skeleton of the boron isotope (NB) tracing drug in the present invention.

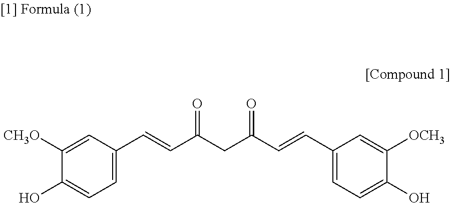

The chemical compound 1 has antitumor activity and carcinogenic preventive effect, but have poor water solubility and as a result show low bioavailability, because it cannot reach a boron concentration enough to ensure the clinical BNCT efficacy of the boron isotope (¹⁰B) tracing drug having wide-area molecular-targeting function and stereotactic destructive power in tumor cells.

For solving this problem, the present invention shows the glycosylation of the phenolic hydroxyl group of the formula (i) to accomplish the enhancement of its water-solubility enough to provide the compound appropriate as boron isotope (¹⁰B) tracing drugs, NBDT drugs, and BNCT drugs. The glycosylation methods are not particularly limited, thus there are available any methods including common reactions such as chemical synthetic methods and tissue culture methods, and microbial methods, but in the present invention, the chemical synthetic method is provided for both the glycosylation reaction and the boron isotope-labeling reaction in terms of yield, purity, and reaction time.

First, the compound 1 according to formula (1), as a lead compound of the present invention, is chemically bonded with a boron isotope (NB) to synthesize a compound 2 according to formula (2). Detailed preparation of the compound 2 from the compound 1 was shown in example 1.

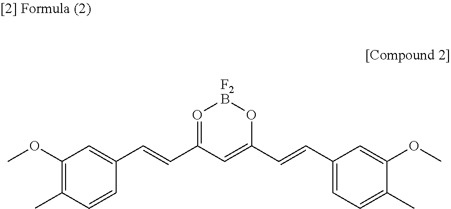

Second, the chemical compound 2, a curcumin-boron isotope (¹⁰B) complex, was glycosylated at its phenolic hydroxyl group to obtain a water-soluble di-glucosylated curcumin-boron isotope (¹⁰B) complex (Chemical compound 3 according to formula (3)), to provide the present invention product tracing drug, as the NBDT drug with its molecular-targeting function and stereotactic destructive power and also as the BNCT drug. Detailed preparation of the compound (3) was shown in example 2.

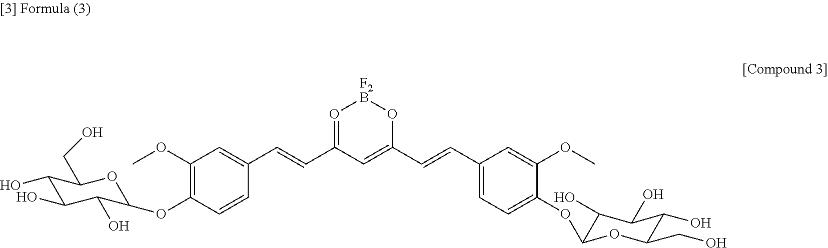

### Effects of the Invention

The present invention provides a new boron isotope (¹⁰B) tracing drug synthesized from the starting material (1E,6E)-1,7-bis(4-hydroxy-3-methoxyphenyl)-1,6-heptadiene-3,5-dione having its standard structure through chemical bonding with the boron isotope (¹⁰B) and then its enhanced water solubility by the glycosylation, which have its high specific molecular traceability and stereotactic destructive power under neutron irradiation and also shows more enhanced bioavailability than the known BNCT drugs with low availabilities. The new invented boron isotope (¹⁰B) tracing drug for NBDT can inhibit tumor cell growth and induce necrosis. The present invention provides a method of introduction of boron isotope (¹⁰B) to the known and common drugs to obtain new boron isotope (¹⁰B) tracing drugs for NBDT and BNCT.

### MODE FOR CARRYING OUT THE INVENTION

The present invention accomplished the preparation of the new drug for NBDT and BNCT from the lead compound 1 through boron isotope (¹⁰B) complex formation and then enhanced water solubility with the glycosylation reaction.

### EXAMPLE 1

### Synthesis of Curcumin-Boron Isotope (¹⁰B) Complex of [Compound 2]

Curcumin (50 mg) was suspended in diethylether (5.0 mL), and boron trifluoride ether (50 μL, 0.407 mmoL) was added dropwise. The reaction mixture was stirred at room temperature for 20 h. The solvent was evaporated under vacuum. The residue was purified by silica gel column chromatography (5% MeOH/CH₂Cl₂ eluent) to give curcumin-boron isotope (¹⁰B) complex (27 mg) [Chemical compound 2]: ¹H-NMR(400 MHz,CH₂Cl₂-d₂): 7.99(d,J=15.4 Hz,2H),7.53-7.61(m,1H), 6.96-7.08 (m,H), 6.56(d.J=15.4 Hz, 2H), 6.02-6.03(s, 6H); HRMS(FAB+)

Calcd. for C₂₁H₁₉BF₂O₆(M+)m/z 416.1243, Found 416.1252.

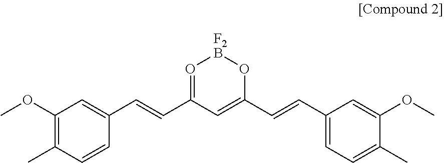

### EXAMPLE 2

The curcumin-boron isotope (¹⁰B) complex obtained in Example 1 (20 mg) was suspended in dimethyl sulfoxide (1.0 mL) and then 0.1 M benzyl triethyl ammonium chloride (1.0 mL) was added under stirring followed by addition of 0.01 M sodium acetate buffer (10 mL). To the reaction mixture, glucose (85.5 mg) and beta-glucosidase (43.2 mg) were added and then incubated at 37.5-45.5+1.0° C. with shaking at 150 rpm for 35 hours. The resulting solution was heated in a boiled water bath for 5-10 minutes and cooled to room temperature. The reaction mixture was extracted with ethyl acetate. The ethyl acetate layers were dried and concentrated under vacuum. The residue was purified by silica gel column chromatography to di-glucosylated curcumin-boron isotope (¹⁰B) complex [Chemical compound 3] in a 25% yield.

¹H-NMR(400 MHz,DMSO-d₆), 3.1-3.4(m, 8H), 3.6(m, 3H), 3.77(s, 6H, OCH₃), 4.31(s, 1H), 4.5(t, 2H, J=5.5 Hz), 4.93(d, 2H J=7 Hz), 4.98(d,2H, J=5 Hz), 5.06(s, 2H), 5.25(d, 2H, J=4 Hz), 6.04(s,1H), 6.81(d, 2H),7.52(d, 2H, J=16 Hz); ESI-MS, m/z715 [M+N]⁺.

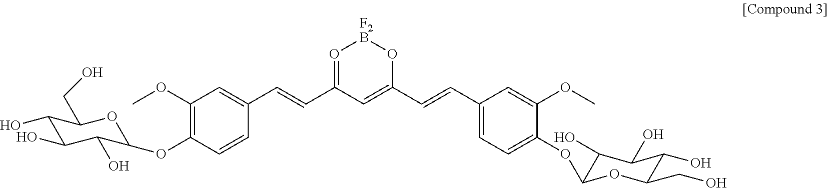

1-4. (canceled)

5. A drug for NBDT, comprising a compound represented by formula (1′) wherein “boron isotope (¹⁰B)” is introduced into a non-active site of (1,6E)-1,7-bis(4-hydroxy-3-methoxyphenyl)-1,6-heptadiene-3,5-dione, provided that boron isotope (¹¹B) is not contained

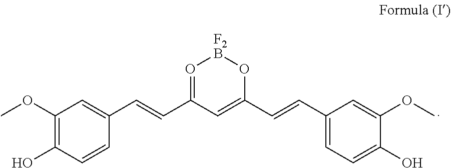

6. A drug for boron neutron capture therapy (BNCT) and a lead compound for NBDT, comprising the compound as claimed in claim 5.

7. The drug for NBDT as claimed in claim 5, wherein the compound of the formula (1′) incorporates a monosaccharide or an oligosaccharide therein to be a boron isotope (¹⁰B) glycoside represented by the following formula (2): 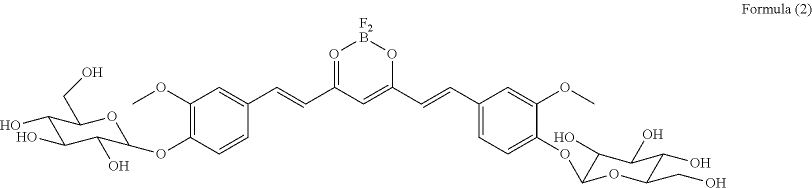
wherein a saccharide residue is a monosaccharide residue or a polysaccharide residue selected from the group consisting of glucosyl, galactosyl, mannosyl, furakutosyl, xylosyl, and arabinosyl groups.

8. A drug for NBDT or BNCT, comprising the boron isotope (10B)-containing curcumin compound according to claim 5.

9. A drug for NBDT or BNCT, comprising the boron isotope (¹⁰B)-containing curcumin glycoside according to claim 7.
